(19) United States
(12) Patent Application Publication
(10) Pub. No.: US 2022/0015659 A1
(43) Pub. Date: Jan. 20, 2022

(54) PROCESSING TIME-FREQUENCY REPRESENTATIONS OF EEG DATA USING NEURAL NETWORKS

(71) Applicant: X Development LLC, Mountain View, CA (US)

(72) Inventors: Mustafa Ispir, Mountain View, CA (US); Asim Iqbal, Zurich (CH); Pramod Gupta, Mountain View, CA (US); Garrett Raymond Honke, Mountain View, CA (US); Vladimir Miskovic, Binghamton, NY (US)

(21) Appl. No.: 16/930,122

(22) Filed: Jul. 15, 2020

(51) Int. Cl. A61B 5/0484; G06N 3/04; G06N 3/08; G06K 9/00; A61B 5/04; G16H 50/30; G16H 50/20

(52) U.S. Cl. CPC A61B 5/0484; G06N 3/0454; G06N 3/08; G16H 50/20; G06K 9/00536; A61B 5/04017; G16H 50/30; G06K 9/0053

(57) ABSTRACT

Methods, systems, and apparatus, including computer programs encoded on computer storage media, for generating embeddings of EEG measurements. One of the methods includes obtaining a two-dimensional time-frequency electroencephalogram (EEG) representation corresponding to one or more EEG signal measurements of a user; processing the time-frequency EEG representation using a first neural network having a plurality of first network parameters to generate an embedding of the time-frequency EEG representation, wherein the first neural network has been trained using transfer learning; and providing the embedding of the time-frequency EEG representation to a downstream neural network to generate a mental health prediction for the user.

### BACKGROUND

This specification relates to generating outputs using neural networks.

Neural networks, or for brevity, networks, are machine learning models that employ multiple layers of operations to predict one or more outputs from one or more inputs. Neural networks typically include one or more hidden layers situated between an input layer and an output layer. The output of each layer is used as input to another layer in the network, e.g., the next hidden layer or the output layer.

Each layer of a neural network specifies one or more transformation operations to be performed on input to the layer. Some neural network layers have operations that are referred to as neurons. Each neuron receives one or more inputs and generates an output that is received by another neural network layer. Often, each neuron receives inputs from other neurons, and each neuron provides an output to one or more other neurons.

An architecture of a neural network specifies what layers are included in the network and their properties, as well as how the neurons of each layer of the network are connected. In other words, the architecture specifies which layers provide their output as input to which other layers and how the output is provided.

The transformation operations of each layer are performed by computers having installed software modules that implement the transformation operations. Thus, a layer being described as performing operations means that the computers implementing the transformation operations of the layer perform the operations.

Each layer generates one or more outputs using the current values of a set of parameters for the layer. Training the neural network thus involves continually performing a forward pass on the input, computing gradient values, and updating the current values for the set of parameters for each layer using the computed gradient values. Once a neural network is trained, the final set of parameter values can be used to make predictions in a production system.

### SUMMARY

This specification describes a system that processes electroencephalogram (EEG) signal measurements to generate an embedding of the EEG signal measurements. In particular, the system can obtain a two-dimensional time-frequency representation of one or more EEG signal measurements and process the time-frequency representation using a first neural network to generate an embedding of the one or more EEG signal measurements represented by the time-frequency representation. In some implementations, the first neural network is a transfer-learned neural network. That is, a training system has trained a second neural network to perform a different image processing task, and the training system used the trained parameters of the second neural network to determine the parameters of the first neural network.

The subject matter described in this specification can be implemented in particular embodiments so as to realize one or more of the following advantages.

Some existing techniques predict whether a user has a mental health disorder by computing the difference between two time-frequency representations of EEG signal measurements corresponding to respective prompts of an EEG task, e.g., pleasant and unpleasant images. Computing a difference between two time-frequency EEG representations might not capture all of the information encoded in the time-frequency EEG representations. Using techniques described in this disclosure, a neural network can automatically learn an optimal embedding of the time-frequency EEG representations that extracts maximal information from the time-frequency representations.

Leveraging a transfer-learned neural network can further allow the system to generate a rich embedding of a time-frequency EEG representation. A pre-trained neural network corresponding to an image processing task can be trained to extract high-level information from images in order to perform the image processing task, e.g., classifying object depicted in the image. This information can also be useful, when extracted from a time-frequency image representation, in predicting the mental health status of a user.

Furthermore, according to implementations of the present disclosure the pre-trained neural network is generally trained on a training data set of images that is larger than available datasets of EEG training data. A system can therefore more efficiently and effectively train a neural network by obtaining parameter values trained on the large training data set of images and fine-tuning the parameter values using a smaller EEG training data set, than if the system attempted to train the neural network on the smaller EEG training data set alone.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a diagram of an example EEG embedding system.

FIG. 2 is an illustration of an example two-dimensional time-frequency representation of EEG signal measurement.

FIG. 3 is a flow diagram of an example process for generating an embedding of a two-dimensional time-frequency EEG representation.

FIG. 4 is a flow diagram of an example process for training a neural network to generate embeddings of two-dimensional time-frequency EEG representations.

Like reference numbers and designations in the various drawings indicate like elements.

### DETAILED DESCRIPTION

This specification describes a system that processes EEG signal measurements to generate an embedding of the EEG signal measurements. The embedding of the EEG signal measurements can be provided to one or more downstream models for generating a mental health prediction of a user.

In this specification, a mental health prediction of a user can be a prediction regarding any aspect of the mental health status of the user. For example, a mental health prediction can represent the likelihood that the user has one or more particular mental illnesses. As another example, a mental health prediction can represent a classification of a personality type of the user, e.g., a prediction that the user belongs to a particular one out of N possible personality types. As another example, the mental health prediction can represent a likelihood that the user will develop one or more illnesses or traits in the future, e.g., a likelihood that the user will develop a particular addiction.

An EEG signal measurement characterizes brain activity of a user. To capture an EEG signal measurement, multiple electrodes are placed on the scalp of the user at different locations, and each electrode measures the electrical activity of the brain at the corresponding location over a period of time. The measurement captured by each electrode is a one-dimensional time series, where each element of the time series represents the amplitude of electrical activity of the brain at the corresponding location at a particular time point. The time series captured by each electrode can be included in a respective channel of the EEG signal measurement. That is, a given EEG signal measurement characterizes the brain activity of the user during a particular period of time and includes one or more channels that each corresponds to a respective location on the scalp of the user.

In some cases, EEG signal measurements are captured while the user is performing a cognitive-behavioral task, referred to in this specification as an “EEG task.” A system can present a prompt corresponding to the EEG task to the user, e.g., using a graphical user interface, and capture an EEG signal measurement in response to the prompt; the process of capturing an EEG signal measurement is referred to in this specification as an “EEG trial.” In some cases, the EEG task can be passive, e.g., the prompt can be to look at an image. In some other cases, the EEG task can be active, e.g., the prompt can be to select a choice from a set of options.

An EEG task can have one or more different prompt types that represent different categories of prompts of the EEG task. The brain activity of the user in response to prompts of different categories will generally be different. As a particular example, an EEG task can be to view an image, a first prompt type can be to view pleasant images, and a second prompt type can be to view unpleasant images. As another example, an EEG task can be to receive real or fake monetary rewards and punishments, e.g., in a gambling context; in this case, a first prompt type can be to view positive monetary reinforcement, and a second prompt type can be to view negative monetary reinforcement.

An EEG signal measurement captured in response to a prompt of a first prompt type and an EEG signal measurement captured in response to a prompt of a second prompt type can be compared in order to generate a mental health prediction of the user. For example, EEG signal measurements corresponding to different respective prompt types can be compared to diagnose one or more mental illnesses, e.g., major depressive disorder, bipolar disorder, or anxiety.

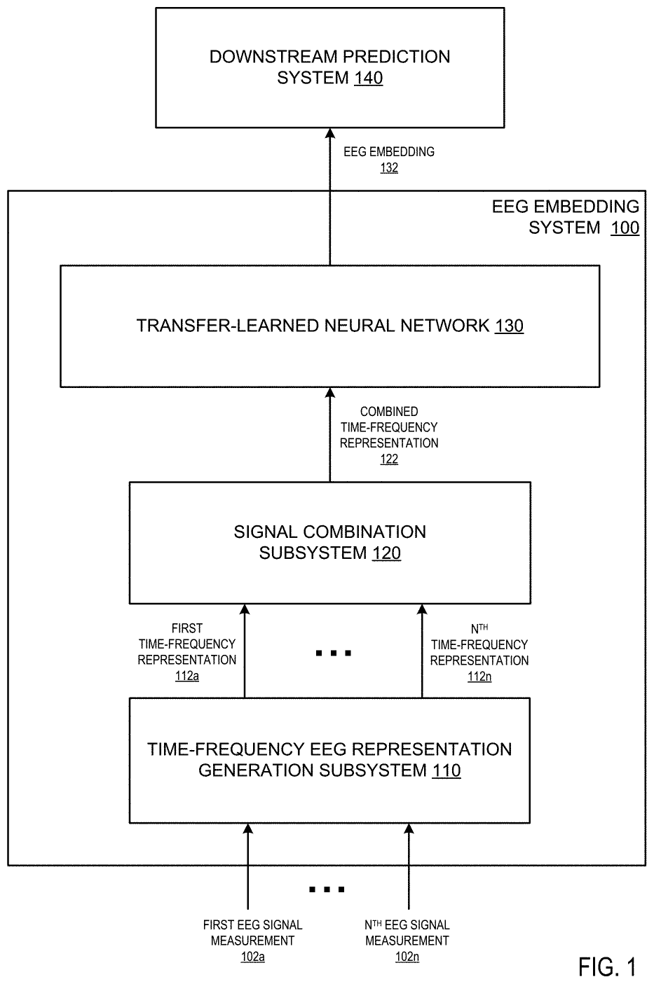

FIG. 1 is a diagram of an example EEG embedding system 100. The EEG embedding system 100 is an example of a system implemented as computer programs on one or more computers in one or more locations, in which the systems, components, and techniques described below can be implemented.

As referred to herein, an embedding is an ordered collection of numeric values that represents an input in a particular embedding space. For example, an embedding can be a vector of floating point or other numeric values that has a fixed dimensionality.

The EEG embedding system 100 receives as input multiple EEG signal measurements 102a-n corresponding to the same user. In some implementations, each of the EEG signal measurements 102a-n corresponding to the same particular prompt type of an EEG task; that is, each EEG signal measurement 102a-n was captured during a respective EEG trial in which the user was presented a prompt of the particular prompt type of the EEG task. The EEG embedding system 100 processes the EEG signal measurements 102a-n to generate an embedding 132 of the EEG signal measurements. That is, the embedding 132 encodes information from each of the EEG signal measurements 102a-n. For example, the embedding 132 can characterize a standard or expected brain activity of the user when presented prompts of the particular prompt type of the EEG task.

Often, an EEG signal measurement corresponding to a single EEG trial can have a high degree of noise. That is, a single EEG signal measurement might not be an accurate representation of the response of the brain of the user to the corresponding prompt type of an EEG task. Therefore, in order to generate an embedding 132 that represents the response of the brain of the user to prompts of the particular prompt type, the signal EEG embedding system 100 combines multiple different EEG signal measurement corresponding to the particular prompt type that were gathered during respective different EEG trials. Thus, because the embedding 132 includes information from multiple different signal measurements gathered during independent EEG trials, the embedding 132 does not have as much noise as a single EEG signal measurement, i.e., is more representative of the response of the brain of the user to the particular prompt type.

The EEG embedding system 100 includes a time-frequency EEG representation generation subsystem 110, a signal combination subsystem 120, and a transfer-learned neural network 130.

The time-frequency EEG representation generation subsystem 110 obtains the EEG signal measurements 102a-n and processes, for each EEG signal measurement, the EEG signal measurement to generate a respective two-dimensional time-frequency EEG representation 112a-n of the EEG signal measurement.

A two-dimensional time-frequency representation of an EEG signal measurement represents, for each time point in the EEG signal measurement, the power spectrum of frequencies of the EEG signal measurement at the time point. The first dimension of the two-dimensional representation is time and includes each time point in the sequence of time points of the EEG signal measurements, and the second dimension of the two-dimensional representation is frequency. The second dimension includes multiple frequencies or multiple ranges of frequencies, i.e., multiple different intervals of consecutive frequencies. That is, the time-frequency representation of an EEG signal measurement characterizes the relative power of multiple different frequencies through time. For instance, each element in the time-frequency representation characterizes the power of the EEG signal measurement of the corresponding frequency at the corresponding time. In implementations in which the EEG signal measurement includes multiple channels each corresponding to a different EEG sensor placed on the scalp of the user, the time-frequency representation can also include multiple channels, where each channel is a time-frequency representation of the measurement corresponding to the respective EEG sensor placed on the scalp of the user.

In some implementations, the time-frequency EEG representation generation subsystem can generate, for each EEG signal measurement 102a-n, the respective time-frequency representation 112a-n by determining the Fourier transform of the EEG signal measurement, e.g., by processing the EEG signal measurement using a fast Fourier Transform (FFT) algorithm.

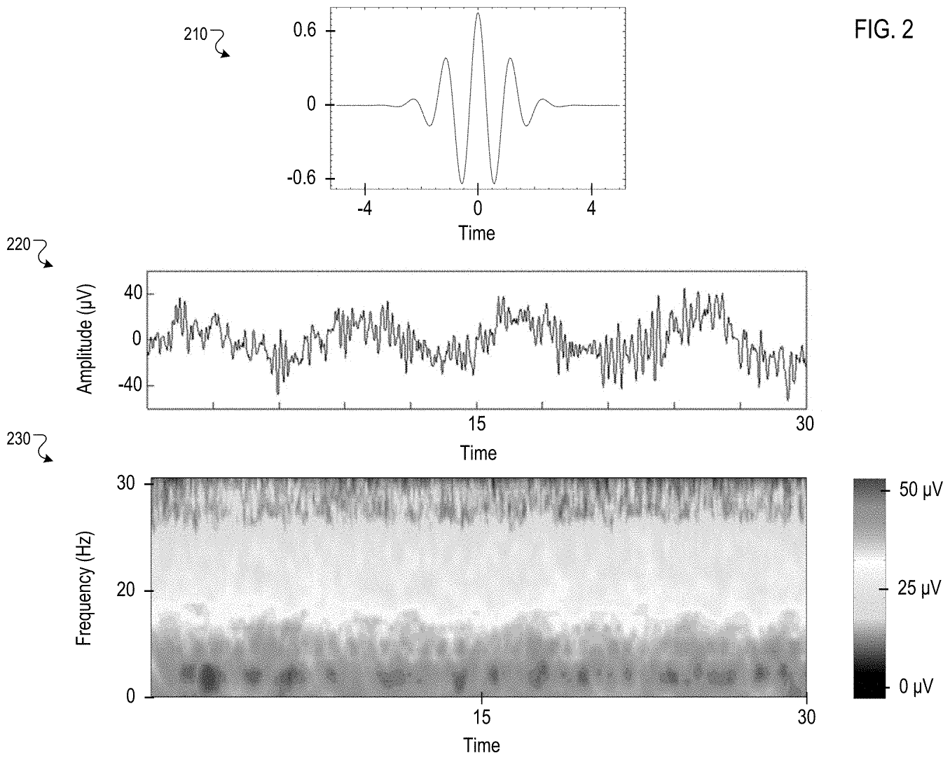

In some other implementations, the time-frequency EEG representation generation subsystem 110 can generate, for each EEG signal measurement 102a-n, the respective time-frequency representation 112a-n using a wavelet transform. That is, subsystem 110 can process the EEG signal measurement 102a-n using multiple versions of a wavelet each corresponding to a different frequency or frequency range. By performing one-dimensional convolution on the EEG signal measurement 102a-n using a version of the wavelet corresponding to a particular frequency, the system can determine the power of the particular frequency at each time point in the EEG signal measurement 102a-n. This process is described in more detail below with respect to FIG. 2.

The time-frequency EEG representation generation subsystem 110 provides the time-frequency representations 112a-n to the signal combination subsystem 120. The signal combination subsystem 110 combines the N time-frequency representations 112a-n corresponding to respective EEG signal measurements 102a-n to generate a combined time-frequency representation 122 that characterizes all of the EEG signal measurements 102a-n. In the implementations, in which each time-frequency representation 112a-n includes multiple channels that each correspond to respective EEG sensors placed on the scalp of the user, the signal combination 120 can combine, for each channel, the channel in each time-frequency representations 112a-n to generate a corresponding channel in the combined time-frequency representation.

In some implementations, the signal combination subsystem 120 determines a mean or median of the n time-frequency representations 112a-n. That is, for each time point and for each frequency in the time-frequency representations 112a-n, the signal combination subsystem 120 can determine the value corresponding to the time point and frequency in the combined time-frequency representation 122 to be the mean or median of corresponding values in the N time-frequency representations 112a-n. In some implementations, when determining the mean or median corresponding to a time point and frequency, the signal combination subsystem 120 can discard one or more outlier values corresponding to respective time-frequency representations 112a-n.

In some other implementations, the signal combination subsystem 120 can process each time-frequency representation 112a-n using a neural network to generate the combined time-frequency representation 122. As a particular example, the signal combination subsystem 120 can process each time-frequency representation 112a-n using an attention-based transformer neural network.

In some implementations, the order of the time-frequency EEG representation generation subsystem 110 and the signal combination subsystem 120 can be reversed. That is, the signal combination subsystem 120 can obtain the EEG signal measurements 102a-n and combine the EEG signal measurements to generate a single combined EEG signal representation. For example, the signal combination subsystem 120 can determine a mean or median EEG signal measurement from the NEEG signal measurements 102a-n. That is, for each time point in the EEG signal measurements 102a-n, the signal combination subsystem 120 can determine the value corresponding to the time point in the combined EEG signal representation to be the mean or median of the corresponding values in the NEEG signal measurements 102a-n. As another example, the signal combination subsystem 120 can process each EEG signal measurement 102a-n using a neural network to generate the combined EEG signal measurement, e.g., an attention-based transformer neural network. Then, the time-frequency EEG representation generation subsystem 110 can obtain the single combined EEG signal representation and process the single combined EEG signal representation to generate the combined time-frequency representation, e.g., by determining the Fourier transform of the combined EEG signal measurement or by processing the combined EEG signal measurement using a wavelet transform.

In other words, in some implementations the EEG embedding system 100 first generates N respective time-frequency representations each corresponding to a respective EEG signal measurement 102a-n, and then combines the N time-frequency representations to generate the combined time-frequency representation 122. In some other implementations, the EEG embedding system 100 first combines the NEEG signal measurements 102a-n to generate a single combined EEG signal measurement, and then generates the combined time-frequency representation 122 from the single combined EEG signal measurement.

In either case, the transfer-learned neural network 130 can obtain the combined time-frequency representation 122 and process the combined time-frequency representation 122 to generate the embedding 132 of the EEG signal measurements 102a-n. In this specification, a neural network is “transfer-learned” if at least some of the parameters of the neural network have been trained, at least in part, by processing training examples to generate respective network outputs related to a different machine learning task. Often, a neural network can learn, when being trained to perform a first machine learning task, to extract information that is useful for a second machine learning task. In this case, the parameters of the transfer-learned neural network 130 can be trained according to a different image processing task, e.g., object detection or semantic segmentation. Thus, the parameters of the network 130 can learn to extract information from images that is useful for executing the different image processing task, and thus to extract useful information from two-dimensional time-frequency EEG representations.

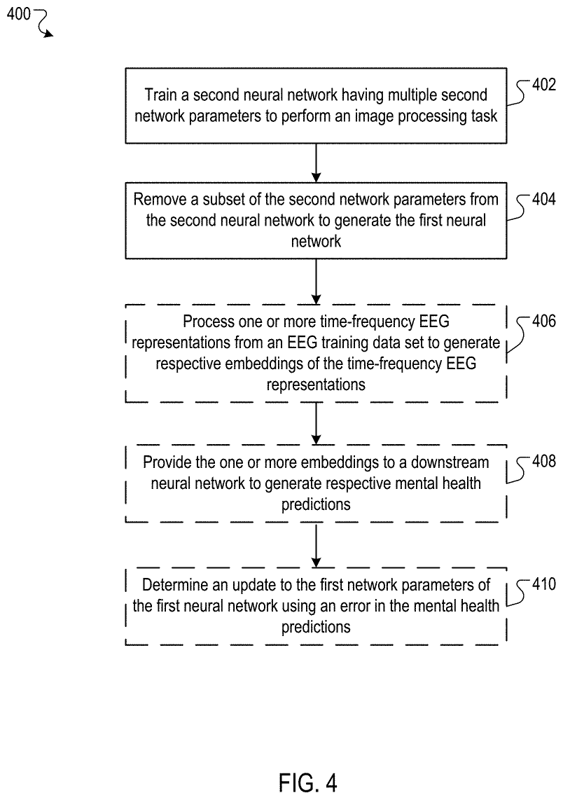

In other words, a training system can train an initial neural network to execute the different image processing task, and then configure transfer-learned neural network 130 using the trained parameters of the initial neural network. For example, the training system can remove one or more layers from the initial neural network to generate the transfer-learned neural network 130. Training transfer-learned neural networks are discussed in more detail below with respect to FIG. 4.

The EEG embedding system 100 can provide the embedding 132 to a downstream prediction system 140 to generate a mental health prediction for the user. For example, the downstream prediction system 140 can be a machine learning model, e.g., a neural network, that processes the embeddings 132 to predict a likelihood that the user has a particular mental health disorder, e.g., major depressive disorder or anxiety. In some implementations, the EEG embedding system 100 can process multiple sets of EEG signal measurements 102a-n that each correspond to a different prompt type of an EEG task, generating a respective embedding 132 for each prompt type of the EEG task. The downstream prediction system 140 can then obtain each embedding 132 corresponding to respective prompt types, and compare the respective embeddings to generate the mental health prediction.

In some implementations, the EEG embedding system 100 can provide the same embedding 132 to multiple different downstream prediction systems 140. For example, the information encoded in the embedding 132 can be useful to generate a prediction for multiple different mental health disorders corresponding to respective downstream systems.

In some implementations, the parameters of the transfer-learned neural network 130 can be fine-tuned jointly with the training of the downstream prediction system 130. That is, a training system can process training example that include EEG signal measurements using the EEG embedding system 100 to generate embeddings of the EEG signal measurements, and then process the embeddings using the downstream prediction system 140 to generate a training prediction. The training system can then determine an error in the training prediction, and backpropagate the error through both the downstream prediction system 140 and the transfer-learned neural network 130 to determine updates to the parameters values of the downstream prediction system 140 and the transfer-learned neural network 130.

FIG. 2 is an illustration of an example two-dimensional time-frequency representation of an EEG signal measurement 220.

A system, e.g., the time-frequency EEG representation generation subsystem 110 depicted in FIG. 1, can process the EEG signal measurement 220 using a wavelet transform to generate the two-dimensional time-frequency representation. The two-dimensional time-frequency representation includes, for each time point in the EEG signal measurement 220, a respective frequency power value for each of multiple frequencies or frequency ranges, and is represented by the spectrogram 230. In this specification, a spectrogram is a visual representation of a two-dimensional time-frequency EEG representation, where different frequency power values are represented by different pixel intensities or colors.

As a particular example, the system can process the EEG signal measurement 220 using multiple different Morletσ wavelets that each correspond to a different frequency. A Morlet wavelet, e.g., the Morlet wavelet 210 illustrated in FIG. 2, is a wavelet composed of a complex exponential multiplied by a Gaussian window. The shape of a Morlet wavelet is defined by a single parameter σ, often called the “number of cycles” of the Morlet wavelet.

For a single parent Morlet wavelet having parameter σ, the system can determine multiple children wavelets having respective frequencies. The system can then convolve each child wavelet along the time dimension of the EEG signal measurement 220 to determine the power of the frequency corresponding to the child wavelet at each time point.

Thus, to generate the time-frequency representation of the EEG signal measurement 220, the system can determine i) the number of cycles a, ii) the frequencies of interest, and ii) a sampling rate. In some implementations, the system can select a different parameter σ for each frequency. In general, a lower number of cycles increases temporal domain precision and a higher number of cycles increases the frequency domain precision of the resulting time-frequency representation.

In some implementations, the system can separately process each of multiple channels of the EEG signal measurement 220 using the Morlet wavelets, where each channel corresponds to a different EEG sensor on the scalp of the user. Thus, the system can generate a respective spectrogram 230 corresponding to each EEG sensor on the scalp of the user.

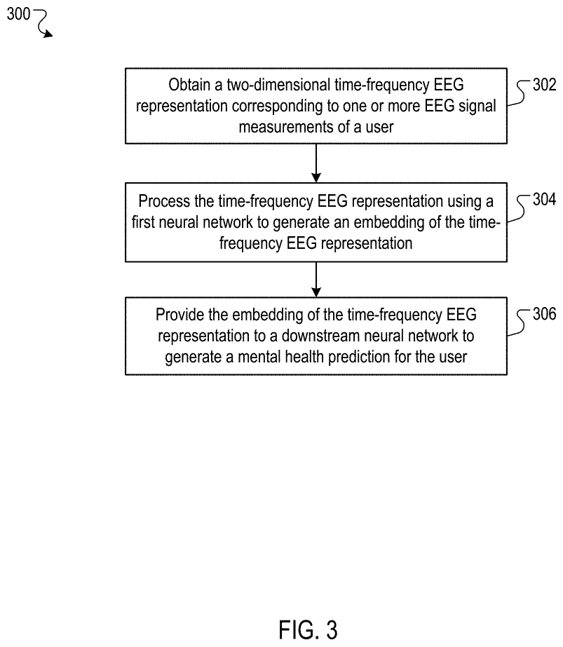

FIG. 3 is a flow diagram of an example process 300 for generating an embedding of a two-dimensional time-frequency EEG representation. For convenience, the process 300 will be described as being performed by a system of one or more computers located in one or more locations. For example, an EEG embedding system, e.g., the EEG embedding system 100 depicted in FIG. 1, appropriately programmed in accordance with this specification, can perform the process 300.

The system obtains a two-dimensional time-frequency EEG representation corresponding to one or more EEG signal measurements of a user (step 302). In some implementations, the two-dimensional time-frequency representation is a combined two-dimensional time-frequency representation corresponding to multiple EEG signal measurements. In some such implementations, the combined two-dimensional time-frequency representation has been generated by obtaining a combined EEG signal measurement corresponding to the multiple EEG signal measurements and generating the combined two-dimensional time-frequency representation using the combined EEG signal measurement. In some other such implementations, the combined two-dimensional time-frequency representation has been generated by obtaining multiple two-dimensional time-frequency representations corresponding to the respective EEG signal measurements and combining the multiple two-dimensional time-frequency representations.

The system processes the time-frequency EEG representation using a first neural network to generate an embedding of the time-frequency EEG representation (step 304). In some implementations, the first neural network has been trained using transfer learning; this process is described in more detail below with respect to FIG. 4.

The system provides the embedding of the time-frequency EEG representation to a downstream neural network to generate a mental health prediction for the user (step 306). In some implementations, the mental health prediction characterizes a likelihood that the user has a particular mental health disorder. In some implementations, the system can provide the network outputs to multiple different downstream neural networks that each process the network outputs to generate respective mental health predictions for the user, e.g., to generate a likelihood that the user has a respective different mental health disorder.

FIG. 4 is a flow diagram of an example process for training a first neural network to generate embeddings of two-dimensional time-frequency EEG representations. The first neural network has multiple first network parameters. For convenience, the process 400 will be described as being performed by a system of one or more computers located in one or more locations. For example, a training system for an EEG embedding system, e.g., the EEG embedding system 100 depicted in FIG. 1, appropriately programmed in accordance with this specification, can perform the process 400.

The system trains a second neural network having multiple second network parameters to perform an image processing task (step 402). That is, the second neural network is configured to process an input that includes an image to generate a corresponding output, e.g., a classification output, a regression output, or a combination thereof. The system can train the second neural network by updating values for the second network parameters using a training data set of training images.

As a particular example, the second neural network can be configured to process an image to generate a classification output that includes a respective score corresponding to each of multiple categories. The score for a category indicates a likelihood that the image belongs to the category. In some cases, the categories may be classes of objects (e.g., dog, cat, person, and the like), and the image may belong to a category if it depicts an object included in the object class corresponding to the category. In some cases, the categories may represent global image properties (e.g., whether the image depicts a scene in the day or at night, or whether the image depicts a scene in the summer or the winter), and the image may belong to the category if it has the global property corresponding to the category.

As another particular example, the second neural network can be configured to process an image to generate a pixel-level classification output that includes, for each pixel, a respective score corresponding to each of multiple categories. For a given pixel, the score for a category indicates a likelihood that pixel belongs to the category. In some cases, the categories may be classes of objects, and a pixel may belong to a category if it is part on an object included in the object class corresponding to the category. That is, the pixel-level classification output may be semantic segmentation output. As a particular example, the second neural network may be an edge detector neural network configured to predict one or more pixels of an image that represent edges of objects depicted in the image.

As another particular example, the second neural network can be configured to process an image to generate a regression output that estimates one or more continuous variables (i.e., that can assume infinitely many possible numerical values) that characterize the image. In a particular example, the regression output may estimate the coordinates of bounding boxes that enclose respective objects depicted in the image. The coordinates of a bounding box may be defined by (x, y) coordinates of the vertices of the bounding box.

The system can use the trained values of the second parameters of the second neural network to determine the values of the first network parameters of the first neural network.

In particular, the system removes a subset of the second network parameters from the second neural network to generate the first neural network (step 404). Each first parameter of the first neural network has the same parameter value as the corresponding second parameters in the second neural network. The first parameters in the first neural network have the same relationships to each other as the corresponding second network parameters in the second neural network. That is, the first neural network has the same network architecture as the second neural network, modulo the parameters that have been removed from the second neural network.

For example, the system can remove one or more final layers of the second neural network to generate the first neural network. As a particular example, if the image processing task is a classification task, then the training system can remove from the second neural network a final softmax layer that processes the final hidden representation and generates the classification predictions; thus, the output of the first neural network would be the final hidden representation, which represents an embedding of the input two-dimensional time-frequency EEG representations.

In some implementations, the system completes the process 400 after step 404. That is, the system determines the final values of the first network parameters to be equal to the trained values of the second neural network.

In some other implementations, the system further trains, i.e., “fine tunes,” the first network parameters by continuing to steps 406-410, in order to improve the embeddings of the time-frequency EEG representations generated by the first neural network.

The system processes one or more time-frequency EEG representations from an EEG training data set using the first neural network to generate respective embeddings of the time-frequency EEG representations (step 406).

The system provides the one or more embeddings to a downstream neural network to generate respective mental health predictions (step 408).

The system determines an update to the first network parameters of the first neural network using an error in the mental health predictions (step 410). For example, the system can backpropagate the error through the downstream neural network to the first neural network to determine the updates to the first network parameters.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

In addition to the embodiments described above, the following embodiments are also innovative:

Embodiment 1 is a method comprising:

obtaining a two-dimensional time-frequency electroencephalogram (EEG) representation corresponding to one or more EEG signal measurements of a user;

processing the time-frequency EEG representation using a first neural network having a plurality of first network parameters to generate an embedding of the time-frequency EEG representation, wherein the first neural network has been trained using transfer learning, the training comprising:
training a second neural network having a plurality of second network parameters to perform an image processing task, comprising updating values for the plurality of second network parameters using a training data set comprising a plurality of training images; and
determining values for the plurality of first network parameters from the trained values of the plurality of second network parameters; and

providing the embedding of the time-frequency EEG representation to a downstream neural network to generate a mental health prediction for the user.

Embodiment 2 is the method of embodiment 1, wherein:

the two-dimensional time-frequency EEG representation is a combined two-dimensional time-frequency EEG representation corresponding to a plurality of EEG signal measurements; and

obtaining the combined two-dimensional time-frequency EEG representation comprises:
obtaining the plurality of EEG signal measurements;
processing each of the plurality of EEG signal measurements to generate respective single-measurement time-frequency EEG representations; and
combining the single-measurement time-frequency EEG representations to generate the combined two-dimensional time-frequency EEG representation.

Embodiment 3 is the method of embodiment 2, wherein combining the single-measurement time-frequency EEG representations comprises determining a mean or median of the single-measurement time-frequency EEG representations.

Embodiment 4 is the method of embodiment 2, wherein combining the single-measurement time-frequency EEG representations comprises processing each single-measurement time-frequency EEG representation using a third neural network to generate the combined two-dimensional time-frequency EEG representation.

Embodiment 5 is the method of any one of embodiments 1-4, wherein obtaining the two-dimensional time-frequency EEG representation corresponding to one or more EEG signal measurements comprises:

obtaining an EEG signal measurement; and

processing the EEG signal measurement using a plurality of wavelets to generate the two-dimensional time-frequency EEG representation, wherein each of the plurality of wavelets corresponds to a different frequency.

Embodiment 6 is the method of any one of embodiments 1-5, wherein determining values for the plurality of first network parameters comprises:

removing a subset of the second network parameters from the second neural network to generate the first neural network, wherein the value for each first network parameter is determined to be equal to a corresponding trained value of the second network parameter.

Embodiment 7 is the method of any one of embodiments 1-5, wherein determining values for the plurality of first network parameters comprises:

removing a subset of the second network parameters from the second neural network to generate the first neural network, wherein an initial value for each first network parameter is determined to be equal to a corresponding trained value of the second network parameter; and

updating the initial values of the plurality of first network parameters using an EEG training data set comprising a plurality of time-frequency EEG representations to generate final values for the plurality of first network parameters.

Embodiment 8 is the method of any one of embodiments 1-7, wherein the two-dimensional time-frequency EEG representation comprises a plurality of two-dimensional channels each corresponding to a different EEG sensor.

Embodiment 9 is the method of any one of embodiments 1-8, wherein each EEG signal measurements corresponds to a same particular prompt of a plurality of prompts of an EEG task.

Embodiment 10 is the method of any one of embodiments 1-9, wherein the mental health prediction characterizes a likelihood that the user has a particular mental health disorder.

Embodiment 11 is the method of embodiment 10, further comprising:

providing the embedding of the time-frequency EEG representation to one or more second downstream neural networks to generate respective second mental health predictions for the user, wherein each second mental health prediction characterizes a likelihood that the user has a respective different mental health disorder.

Embodiment 12 is a system comprising: one or more computers and one or more storage devices storing instructions that are operable, when executed by the one or more computers, to cause the one or more computers to perform the method of any one of embodiments 1 to 11.

Embodiment 13 is a computer storage medium encoded with a computer program, the program comprising instructions that are operable, when executed by data processing apparatus, to cause the data processing apparatus to perform the method of any one of embodiments 1 to 11.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
obtaining a two-dimensional time-frequency electroencephalogram (EEG) representation corresponding to one or more EEG signal measurements of a user;
processing the time-frequency EEG representation using a first neural network having a plurality of first network parameters to generate an embedding of the time-frequency EEG representation, wherein the first neural network has been trained using transfer learning, the training comprising:
training a second neural network having a plurality of second network parameters to perform an image processing task, comprising updating values for the plurality of second network parameters using a training data set comprising a plurality of training images; and
determining values for the plurality of first network parameters from the trained values of the plurality of second network parameters; and
providing the embedding of the time-frequency EEG representation to a downstream neural network to generate a mental health prediction for the user.

2. The method of claim 1, wherein:
the two-dimensional time-frequency EEG representation is a combined two-dimensional time-frequency EEG representation corresponding to a plurality of EEG signal measurements; and
obtaining the combined two-dimensional time-frequency EEG representation comprises:
obtaining the plurality of EEG signal measurements;
processing each of the plurality of EEG signal measurements to generate respective single-measurement time-frequency EEG representations; and
combining the single-measurement time-frequency EEG representations to generate the combined two-dimensional time-frequency EEG representation.

3. The method of claim 2, wherein combining the single-measurement time-frequency EEG representations comprises determining a mean or median of the single-measurement time-frequency EEG representations.

4. The method of claim 2, wherein combining the single-measurement time-frequency EEG representations comprises processing each single-measurement time-frequency EEG representation using a third neural network to generate the combined two-dimensional time-frequency EEG representation.

5. The method of claim 1, wherein obtaining the two-dimensional time-frequency EEG representation corresponding to one or more EEG signal measurements comprises:
obtaining an EEG signal measurement; and
processing the EEG signal measurement using a plurality of wavelets to generate the two-dimensional time-frequency EEG representation, wherein each of the plurality of wavelets corresponds to a different frequency.

6. The method of claim 1, wherein determining values for the plurality of first network parameters comprises:
removing a subset of the second network parameters from the second neural network to generate the first neural network, wherein the value for each first network parameter is determined to be equal to a corresponding trained value of the second network parameter.

7. The method of claim 1, wherein determining values for the plurality of first network parameters comprises:
removing a subset of the second network parameters from the second neural network to generate the first neural network, wherein an initial value for each first network parameter is determined to be equal to a corresponding trained value of the second network parameter; and
updating the initial values of the plurality of first network parameters using an EEG training data set comprising a plurality of time-frequency EEG representations to generate final values for the plurality of first network parameters.

8. The method of claim 1, wherein the two-dimensional time-frequency EEG representation comprises a plurality of two-dimensional channels each corresponding to a different EEG sensor.

9. The method of claim 1, wherein each EEG signal measurements corresponds to a same particular prompt of a plurality of prompts of an EEG task.

10. The method of claim 1, wherein the mental health prediction characterizes a likelihood that the user has a particular mental health disorder.

11. The method of claim 10, further comprising:
providing the embedding of the time-frequency EEG representation to one or more second downstream neural networks to generate respective second mental health predictions for the user, wherein each second mental health prediction characterizes a likelihood that the user has a respective different mental health disorder.

12. A system comprising one or more computers and one or more storage devices storing instructions that are operable, when executed by the one or more computers, to cause the one or more computers to perform a method comprising:
obtaining a two-dimensional time-frequency electroencephalogram (EEG) representation corresponding to one or more EEG signal measurements of a user;
processing the time-frequency EEG representation using a first neural network having a plurality of first network parameters to generate an embedding of the time-frequency EEG representation, wherein the first neural network has been trained using transfer learning, the training comprising:
training a second neural network having a plurality of second network parameters to perform an image processing task, comprising updating values for the plurality of second network parameters using a training data set comprising a plurality of training images; and
determining values for the plurality of first network parameters from the trained values of the plurality of second network parameters; and
providing the embedding of the time-frequency EEG representation to a downstream neural network to generate a mental health prediction for the user.

13. The system of claim 12, wherein:
the two-dimensional time-frequency EEG representation is a combined two-dimensional time-frequency EEG representation corresponding to a plurality of EEG signal measurements; and
obtaining the combined two-dimensional time-frequency EEG representation comprises:
obtaining the plurality of EEG signal measurements;
processing each of the plurality of EEG signal measurements to generate respective single-measurement time-frequency EEG representations; and
combining the single-measurement time-frequency EEG representations to generate the combined two-dimensional time-frequency EEG representation.

14. The system of claim 12, wherein obtaining the two-dimensional time-frequency EEG representation corresponding to one or more EEG signal measurements comprises:
obtaining an EEG signal measurement; and
processing the EEG signal measurement using a plurality of wavelets to generate the two-dimensional time-frequency EEG representation, wherein each of the plurality of wavelets corresponds to a different frequency.

15. The system of claim 12, wherein determining values for the plurality of first network parameters comprises:
removing a subset of the second network parameters from the second neural network to generate the first neural network, wherein the value for each first network parameter is determined to be equal to a corresponding trained value of the second network parameter.

16. The system of claim 12, wherein determining values for the plurality of first network parameters comprises:
removing a subset of the second network parameters from the second neural network to generate the first neural network, wherein an initial value for each first network parameter is determined to be equal to a corresponding trained value of the second network parameter; and
updating the initial values of the plurality of first network parameters using an EEG training data set comprising a plurality of time-frequency EEG representations to generate final values for the plurality of first network parameters.

17. One or more non-transitory computer storage media encoded with computer program instructions that when executed by a plurality of computers cause the plurality of computers to perform operations comprising:
obtaining a two-dimensional time-frequency electroencephalogram (EEG) representation corresponding to one or more EEG signal measurements of a user;
processing the time-frequency EEG representation using a first neural network having a plurality of first network parameters to generate an embedding of the time-frequency EEG representation, wherein the first neural network has been trained using transfer learning, the training comprising:
training a second neural network having a plurality of second network parameters to perform an image processing task, comprising updating values for the plurality of second network parameters using a training data set comprising a plurality of training images; and
determining values for the plurality of first network parameters from the trained values of the plurality of second network parameters; and
providing the embedding of the time-frequency EEG representation to a downstream neural network to generate a mental health prediction for the user.

18. The non-transitory computer storage media of claim 17, wherein:
the two-dimensional time-frequency EEG representation is a combined two-dimensional time-frequency EEG representation corresponding to a plurality of EEG signal measurements; and
obtaining the combined two-dimensional time-frequency EEG representation comprises:
obtaining the plurality of EEG signal measurements;
processing each of the plurality of EEG signal measurements to generate respective single-measurement time-frequency EEG representations; and
combining the single-measurement time-frequency EEG representations to generate the combined two-dimensional time-frequency EEG representation.

19. The non-transitory computer storage media of claim 17, wherein obtaining the two-dimensional time-frequency EEG representation corresponding to one or more EEG signal measurements comprises:
obtaining an EEG signal measurement; and
processing the EEG signal measurement using a plurality of wavelets to generate the two-dimensional time-frequency EEG representation, wherein each of the plurality of wavelets corresponds to a different frequency.

20. The non-transitory computer storage media of claim 17, wherein determining values for the plurality of first network parameters comprises:
removing a subset of the second network parameters from the second neural network to generate the first neural network, wherein an initial value for each first network parameter is determined to be equal to a corresponding trained value of the second network parameter; and
updating the initial values of the plurality of first network parameters using an EEG training data set comprising a plurality of time-frequency EEG representations to generate final values for the plurality of first network parameters.
